(19) United States
(12) Patent Application Publication
(10) Pub. No.: US 2014/0094873 A1
(43) Pub. Date: Apr. 3, 2014

(54) FOOTWEAR PRODUCT FOR FUNCTIONAL ELECTRICAL STIMULATION

(71) Applicants: Jonas Emborg, Stenlose (DK); Ole Kæseler Andersen, Skorping (DK); Erika Geraldina Spaich, Klarup (DK)

(72) Inventors: Jonas Emborg, Stenlose (DK); Ole Kæseler Andersen, Skorping (DK); Erika Geraldina Spaich, Klarup (DK)

(73) Assignee: NORDIC NEUROSTIM APS, Aalborg Ø (DK)

(21) Appl. No.: 14/116,633

(22) PCT Filed: May 15, 2012
(86) PCT No.: PCT/DK2012/050171

§ 371 Date: Nov. 8, 2013

(30) Foreign Application Priority Data
May 17, 2011 (DK) PA 2011 70244

(51) Int. Cl. A61N 1/36

(52) U.S. Cl. CPC A61N 1/36003
USPC 607/49

(57) ABSTRACT

A product, e.g. an insole for a shoe, fitting onto a person's foot sole based on a platform (SM) of a flexible and electrically isolating material. Multiple stimulation electrodes (S1, S2, S3) with adhesive stimulation areas of at least 1 cm² are spatially distributed on the platform (SM). A conducting and adhesive ground plane electrode (G) with at least an area of 10 times a stimulation electrode area is arranged for contacting the person's foot. An electric interface (I) comprising a plug or socket with externally accessible electrical connectors to allow external individual electrical access to the plurality of stimulation electrode areas (S1, S2, S3) and to the ground plane electrode (G). The electric interface is capable of handling at least a voltage of 200 V, preferably 400 V. Such product is suited for reflex-based gait therapy, where the person's withdrawal reflex is triggered by a painful stimulation of the person's foot sole. Many distributed stimulation electrodes allow individual fitting for optimal stimulation, and low cost sensors built into the product may be used to provide feedback regarding the gait phase, i.e. the product is a one-time use insole suited for easy and hygienic gait therapy in a clinic.

### FIELD OF THE INVENTION

The invention relates to the field of medical devices, specifically the invention provides a footwear product for use in Functional Electrical Stimulation (FES) or Functional Electrical Therapy (FET).

### BACKGROUND OF THE INVENTION

Functional Electric Stimulation FES or Functional Electric Therapy FET is well known to assist patients in walking. For example the Ph.D. thesis “Modulation of the nociceptive withdrawal reflex and its use in rehabilitation of gait of stroke patients”, Aalborg University 2009, by one of the inventors, Jonas Emborg describes reflex-based gait training where a painful electric stimulation on the patient's foot sole helps the patient to move his/her leg and thus initiate a step due to the evoked withdrawal reflex. This provides a helpful tool to enable gait training of patients being partially or completely immobile due to a hemiparetic condition caused by a stroke or traumatic brain injury which has resulted in damage of an area of their brain. Such patients have a significant better chance of rehabilitation if intensive gait training is initiated within 3 months after the stroke. In this early period the brain is especially suited to regenerate/relearn the ability to control the muscles, if the sufficient sensory, learning input is provided, i.e. by completing functionally adequate gait training.

Further references from the inventors are:

“Design and test of a novel closed-loop system that exploits the nociceptive withdrawal reflex for swing phase support of the hemiparetic gait”, J. Emborg, Z. Matja\check{c}ić, J. D. Bendtsen, E. G. Spaich, I. Cikajlo, N. Goljar, O.K. Andersen. IEEE Transactions on Biomedical Engineering. vol. 58, no. 4, pp. 960-970, April 2011. DOI:10.1109/TBME.2010.2096507

“Withdrawal reflex responses evoked by repetitive painful stimulation delivered on the sole of the foot during late stance: site, phase, and frequency modulation”, E. G. Spaich, J. Emborg, T. Collet, L. Arendt-Nielsen, and O. K. Andersen, Exp. Brain Res., vol. 194, no. 3, pp. 359-368, April 2009

“Withdrawal reflexes examined during human gait by ground reaction forces: site and gait phase dependency”, J. Emborg, E. G. Spaich, and O. K. Andersen, Med. Biol. Eng. Comput. 2009; vol. 4, pp. 29-39, January 2009

“Novel method exploiting the nociceptive withdrawal reflexes in rehabilitation of hemiplegic gait” Emborg, J.; Bendtsen, J. D.; Spaich, E. G.; Andersen, O. K., 2009. s. 84-87 World Congress on Medical Physics and Biomedical Engineering, Munich, Germany, 7-12 Sep. 2009, International Federation for Medical and Biological Engineering Proceedings. 25. IX

Laboratory experiments with the above-mentioned reflex-based gait training can be performed using electric stimulation applied by means of adhesive electrodes manually attached to the patient's foot, e.g. electrodes as used for ECG monitoring can be used. However, for practical application of the method several electrodes are used, and thus in a physiotherapy clinic or at a hospital, time is wasted on mounting and taking off the equipment instead of providing actual gait training for a patient.

### SUMMARY OF THE INVENTION

Therefore, following the above description, there is a need for a product to facilitate practical and easy application of painful electrical stimulations of the person's foot for reflex-based gait training.

In a first aspect, the invention provides a product shaped to fit onto a person's foot sole, the product comprising

a platform of a flexible and electrically isolating material,

a plurality of separate stimulation electrodes each having an adhesive stimulation area of at least 1 cm², wherein the stimulation electrodes are spatially distributed on the platform so as to be able to stimulate different zones of the person's foot sole during normal use,

at least one electrically conducting and adhesive ground plane electrode for contacting the person's foot, wherein a total area of the ground plane electrode is at least 10 times an area of one stimulation electrode,

an electric interface comprising a plug or socket with externally accessible electrical connectors so as to allow external individual electrical access to the plurality of stimulation electrode areas and to the ground plane electrode, and

a set of electrical conductors for interconnecting the plurality of stimulation electrodes and the ground plane electrode with the electric connectors of the electric interface, wherein the set of electrical conductors are arranged to withstand a voltage of more than 200 V, e.g. more than 400 V, between the ground plane electrode and the plurality of stimulation electrodes.

Such product, e.g. in the form of a disposable sole for a shoe, is suitable in practical gait training therapy because the stimulation equipment necessary only needs to be plugged onto the electric interface of such product to initiate a gait therapy session. Time consuming individual matching and mounting of stimulation electrodes, often several electrodes, is eliminated, since the product can be manufactured in different sizes thus fitting onto the person's foot without any individual electrode positioning considerations. The product needs to be mounted on the foot sole of the person, and the electric interface needs to be connected to the control system serving to apply the stimulation signal, e.g. in the form of a portable control device carried in the person's belt or the like which may then be wired or wirelessly connected to a stationary or laptop computer system. It is possible to manufacture the product at a low price, thus allowing the product to be used only once, thereby ensuring a high hygienic level without the need for cleaning of the equipment between two persons using the gait training equipment.

The invention can be seen as a product specific to be used primarily, but not exclusively, in connection with the Functional Electric Therapy, FET. The technology has also potential for being used in relation to a diagnostic tool that quantifies central sensitization in relation to chronic pain. This has the application to diagnose chronic pain patients—a large and until now unsolved problem—by assessing expanded reflex receptive fields via distributed electrical stimulations. Stimulations are delivered to different sites on the sole of the foot with the intent to assess spatial variation in withdrawal reflex sensitivity. Also, here a practical method of mounting several electrodes are needed to reduce the time for donning and doffing equipment.

Furthermore, in embodiments with many spatially separated stimulation electrodes, the best possible electrical stimulation of the individual person becomes possible irrespective of individual spatial differences in persons' sensitivities. The computer algorithm can take into account utilization of the best possible stimulation positions based on a feedback loop indicating the resulting swing of the leg and activating the most appropriate stimulation electrodes accordingly. The fact that the product is capable of handling 200 V, preferably also more than 400 V, or more, allows for efficient powerful stimulation, and the defined area difference between ground plane electrode and stimulation electrodes ensures that the intentional withdrawal reflex provoking sensation is obtained when a suitable electric stimulation signal is applied.

In versions with a special electric interface plug, a high safety can be obtained since the risk is minimized that wrong equipment is connected to the product.

The product may be in the form of a more or less simple footwear product, and especially the product may be: 1) a replaceable insole arranged to fit into a shoe, 2) a slipper, or 3) a shoe. The platform may form part of a footwear, such as a shoe, or a slipper.

In one embodiment, the product is a replaceable insole arranged to fit into a person's shoe, and wherein the platform comprises an upper layer and a lower layer with the set of electrical conductors arranged between the upper and lower layers, and wherein the stimulation electrodes extend through holes in the upper layer. As explained, such product can be manufactured at a low cost thus allowing one-time use and thereby easy mounting of a new clean product on the foot of each person. Especially, at least the upper layer may be made of a reusable material, such as a sheet of nonwoven material. Such embodiment will allow only a limited waste of material in case of a one-time use product.

In some embodiments, at least one stimulation electrode is spatially distributed on the platform so as to enable stimulation of each one of the person's foot sole areas: heel, fore foot, and mid foot. This allows a FET control system to activate the most efficient stimulation positions for obtaining the desired withdrawal reflex and thus e.g. reduce adaptation (habituation) effects. More specifically, it may be preferred that the product comprises a plurality of stimulation electrodes spatially distributed so as to enable stimulation of a plurality of different positions of each one of the person's foot sole areas: heel, fore foot, and mid foot. As already explained, this allows an automated FET system to activate the most appropriate positions according to a feedback loop, thus accounting for individual differences.

In some embodiments, the ground plane extends spatially so as to enable contact to at least one of the person's foot regions: heel of foot sole, fore foot of foot sole, mid foot of foot sole, back of heel, and upper part of foot. It may be preferred that the ground plane electrode extends spatially so as to enable contact to a plurality of said regions of the person's foot. In one specific embodiment, the product comprises a plurality of spatially separated ground plane electrodes, while in other embodiments, the ground plane electrode may extend in several ones of said regions by constituting one large conductive area.

It may be preferred that the platform is substantially foot shaped, such that it fits the shape of a normal foot and thus fits inside existing footwear, in case it is an insole product.

In some embodiments, the product comprises an additional platform element structurally linked to the platform, wherein the additional platform element comprises a stimulation electrode or a ground plane electrode in electric connection with said electric interface. Especially, the additional platform element is connected to the platform so as to enable the stimulation electrode or the ground plane electrode to be in contact with an upper part of or a back of the heel of the person's foot when mounted for normal use. Such additional platform element may be in the form of a flap that can be bent to make contact to the back of the heel or the upper part of the person's foot during normal use.

The product may comprise a sensor arranged to provide a feedback indicative of a person's gait phase upon the person walking when wearing the product, such as a sensor arranged between the platform and an upper layer of the product. Such sensor will allow the product to be used in FET based systems with feedback, where the sensor can be used to provide feedback to the FET algorithm regarding in which gait phase the leg is. This may eliminate the need for further feedback sensors, thus providing easy mounting of the equipment, since no individual fitting is needed, if the sensor is integrated in the product. In specific embodiment, the sensor is electrically connected to one or more electrical connectors within said electric interface, such as within the same plug or socket providing the external connection of the stimulation electrodes and the ground plane. This allows very easy connection to a feedback based FET system. Especially, it may be preferred that at least one sensor is spatially arranged in relation to the platform so as to enable sensing of the person's foot touching the ground, when wearing the product. The sensor may comprise an electric switch arranged to switch between an electrically closed and electrically open state upon a person's foot touching the ground, when wearing the product. Such electric switch may be implemented by one of: a force sensitive sensor, an electric switch with an on state and an off state, capacitive transducers, load cells, strain gauges, piezoelectrical force sensors. Simple sensors can be manufactured in low cost and still allow the product to be in the form of a one-time use insole product. The sensor may include one or more advanced sensor technologies, e.g. a laser-based distance sensor to sense distance to the ground, an accelerometer etc., however in such case the manufacturing costs will increase and thus such embodiments are not suited for one-time use.

To further allow a more detailed information about the gait phase, the sensor may comprise a plurality of spatially distributed sensor elements, such as a plurality of electric switches, in different regions of the platform so as to enable sensing of respective areas of the person's foot touching the ground, when wearing the product. Especially, the plurality of sensor elements are electrically connected to respective electrical connectors of the electric interface so as to allow external individual electrical access to the plurality of sensor elements via one single plug or socket of the electric interface.

The electric interface may be implemented by a connector in the form of a plug or socket having a shape different from standard connectors, so as to avoid non-intended connection to external equipment.

The set of electrical conductors may comprise a flexible PCB spatially extending within the platform so as to electrically connect each of the plurality of stimulation electrodes and the respective electrical connectors of the electric interface.

An end of the set of electrical conductors may extend away from the platform, so as to allow the plug or socket of the electric interface to be placed outside a person's shoe.

In a second aspect, the invention provides a system arranged for gait training, such as for rehabilitation of a person after a stroke, the system comprising
a product according to the first aspect,
a stimulation unit arranged for connection to the electric interface of the product, wherein the stimulation unit is arranged to generate an electric stimulation voltage between the ground plane electrode and at least one of the stimulation electrodes, wherein the electric stimulation voltage is high enough to induce a pain reaction on a person wearing the product.

In one embodiment, the system comprises

a sensor arranged to sense a parameter representative of a movement of the person's leg and to generate a feedback signal accordingly, such as a sensor as described in embodiments of the first aspect, and

a processor unit operationally connected to the stimulation unit and to the sensor, the processor unit comprising a processor running a control algorithm so as to generate a control signal to the stimulation unit in response to the feedback signal.

In a third aspect, the invention provides use of a product according to the first aspect for Functional Electric Stimulation, such as reflex based gait training, such as for rehabilitation of a person after a stroke.

In a fourth aspect, the invention provides use of a product according to the first aspect for diagnostics of central sensitization in relation to chronic pain by assessing expanded reflex receptive fields via distributed electrical stimulation.

It is appreciated that the same advantages and equivalent embodiments apply for the second and third aspects as mentioned for the first aspect.

### BRIEF DESCRIPTION OF THE DRAWINGS

Embodiments of the invention will be described, by way of example only, with reference to the drawings, in which

FIG. 1 illustrates an example of stimulation electrode and ground plane electrode layout of an insole embodiment,

FIGS. 2a and 2b illustrate two views of an insole embodiment with a ground plane electrode and a heel stimulation electrode positioned on flaps on the insole platform,

FIGS. 3a and 3b illustrate two views of another insole embodiment with one centrally positioned ground plane electrode,

FIG. 4 illustrates basic parts of a FET system including a product of the present invention,

FIG. 5 illustrates an insole embodiment connected to parts of a FET system, and

FIG. 6 illustrates an example of an implementation of a FET system including an insole according to the invention.

### DESCRIPTION OF EMBODIMENTS

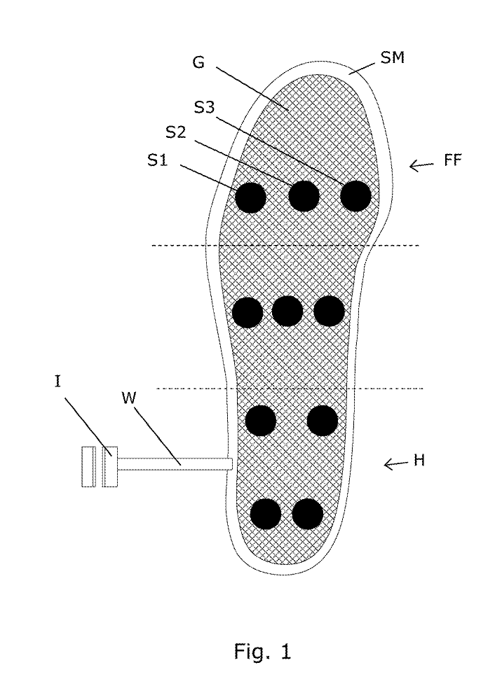

FIG. 1 illustrates the layout of an insole embodiment. A foot shaped platform SM of a sole material such as rubber, polymer, plastic, shock-absorbing synthetic foam etc. which forms the basis of the insole. On top of the platform SM, the black areas indicate stimulation electrodes S1, S2, S3, here shown in a version with a total of 10 stimulation electrodes S1, S2, S3. A ground plane electrode G is indicated as a crossed area, and even though not clear on the sketch, it is understood that the stimulation electrodes S1, S2, S3 are electrically insulated from the ground plane electrode G. To provide good electrical contact with the skin on the foot sole of the person wearing the insole, the ground plane electrode G as well the stimulation electrodes S1, S2, S3 have adhesive surfaces, such as also known from ECG electrodes and the like. Not visible electrical conducting elements ending in a bundle of wires W serve to electrically connect the stimulation electrodes S1, S2, S3 and the ground plane electrode G to respective pins of the single electrical interface plug I, thus allowing easy connection to stimulation control equipment using only one common interface plug I for all electrodes G, S1, S2, S3 that can thus be individually controlled via the plug I. Of course electrode materials, connectors, wires W, and interface plugs I and electrical insulation materials should be able to withstand at least 200 V, preferably at lest 400 V, in order to manage the rather high voltages than can be required for providing stimulation current for reflex-based stimulation.

It appears that the area covered by the ground plane electrode G is significantly larger than the area covered by each of the stimulation electrodes S1, S2, S3, and in the present embodiment the ground plane electrode G covers the majority of the platform area, in fact more than 90% of the platform area, and it is formed as one single area that extends in both forefoot, arch of foot, and heel areas. The large area compared to the stimulation electrode area is required to provide the desired painful sensation provoking a withdrawal effect, which is the basis for a reflex-based gait training FET system. Preferably a ratio between area of ground plane electrode and area of one stimulation electrode is more than 10, such as a ratio of 15-20. For cases where the ground plane electrode extends over a large area of the foot, the ratio may be up to such as 50 or even 100. Further, the area of one stimulation electrode is preferably more than 1 cm², such as 1-2 cm², or such as 2-5 cm².

As seen in the layout sketch of FIG. 1, there are groups of a plurality of stimulation electrodes in each of the foot regions: forefoot FF, arch of the foot, and heel H. This spatial distribution of individually addressable stimulation areas will provide a flexible fitting to each person's response, thus the insole is suited for use with a highly automated FET system that is capable of receiving feedback and select the most appropriate stimulation area in response to feedback regarding the movement of the person's leg. Thus, with many distributed stimulation electrodes S1, S2, S3, a good performance can be achieved without the need for individual electrode positioning for each person.

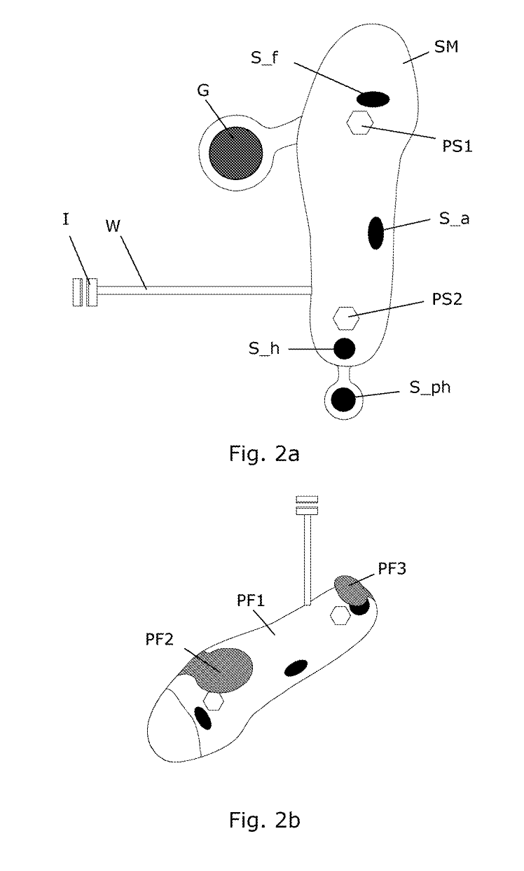

FIGS. 2a and 2b illustrate an alternative insole embodiment. Here the platform SM is also foot shaped, but in the illustrated layout only one stimulation electrode is present in each of the foot sole regions, namely a fore foot electrode S_f, an arch of the foot electrode S_a, and a heel electrode S_h on the main part of the platform PF1. In addition, a flap PF3 is attached to the heel part of the main platform part PF1, e.g. integrally manufactured, e.g. monolithically integrated, with the platform SM material, and carries a stimulation electrode S_ph which fits to the posterior part of the person's heel when flapped into place for normal use as is seen in FIG. 2b. In a similar manner a flap PF2 is attached to the main part of the platform PF1. This flap PF2 carries the ground plane electrode G which fits to contact an upper part of the person's foot when flapped into place for normal use. Thus, this embodiment allows stimulation also of the posterior part of the person's heel in addition to the three foot sole regions.

Further, FIGS. 2a and 2b illustrate the presence of sensors in the form of two pressure sensitive areas, namely one on the forefoot PS1, and one on the heel PS2. Such pressure sensitive areas can be implemented in various ways as known to the skilled person. The function of the sensors PS1, PS2 is to allow feedback indicative of the position of the person's foot during a step, i.e. primarily to know if part of the person's foot touches the ground or not, and in this way discriminate between gait phases.

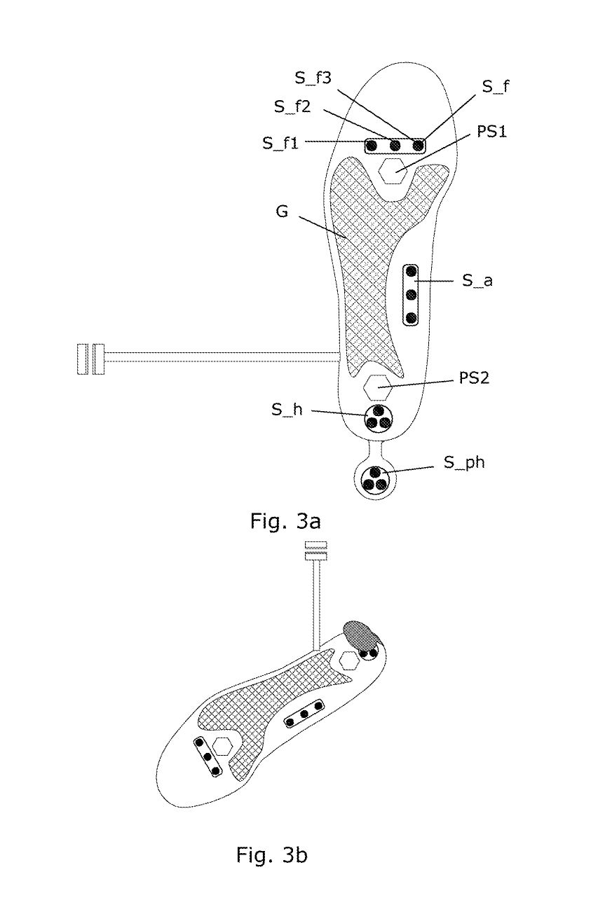

FIGS. 3a and 3b illustrate yet another insole embodiment, referring to details in the foregoing embodiments already described. Again a foot shaped platform forms the basis onto which groups of stimulation electrodes, namely one group S_f of three stimulation electrodes S_f1, S_f2, S_f3 in the forefoot region, one group S_a in the arch of foot region, one group in the heel region S_h, and one group S_ph on a heel flap arranged for contact with a posterior part of the person's heel. In addition, two pressure sensitive sensors are positioned in each part of the foot sole, namely one in the forefoot region PS1, and one in the heel region PS2. One large ground plane electrode G extends primarily in the arch of the foot region, and only partly in the forefoot and heel regions. As already mentioned, the groups of a plurality of stimulation electrodes each individually accessible via the electric interface allows for individual adjustment and refining of stimulation, e.g. based on an automated algorithm using feedback from the sensors PS1, PS2.

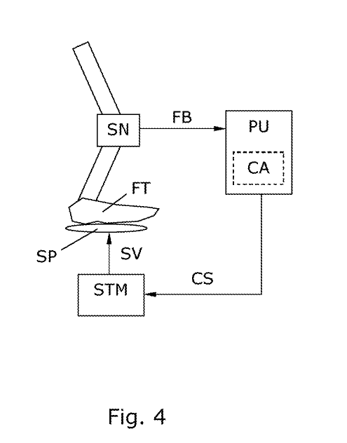

FIG. 4 shows schematically the leg and foot FT of a person together with basic parts of a FET or FES therapy system including a product SP according to the invention. The system includes a stimulator unit STM arranged to apply a stimulation voltage SV to at least a ground plane electrode terminal and a stimulation electrode terminal of the electric interface of the insole or shoe wear product SP, e.g. in the form of insoles as described in the foregoing. The electrodes of the product SP then accordingly induce a painful sensation on the person's foot FT with the purpose of provoking the person's withdrawal reflex. This reflex results in a withdrawal of the leg, i.e. the leg will initiate a swing phase.

The stimulation voltage SV required, e.g. typically up to voltages within the range 50-400 V though typically around 200 V, in order to generate the required stimulation current to trigger the withdrawal reflex, is applied by the stimulator unit STM which is controlled in response to a control signal CS generated by a processor unit PU which runs a control algorithm CA. This control algorithm CA calculated the control signal CS in response to a feedback signal FB which the processor unit PU receives from a sensor SN which is arranged to sense a parameter, e.g. acceleration of the leg, here illustrated as a goniometer SN sensing the knee angle, and generates the feedback signal FB according to this parameter. The control algorithm CA is preferably designed to adjust the control signal CS so as to obtain a target trajectory for the leg. In the illustrated example, the control algorithm CA may be designed to determine a control signal CS resulting in a stimulation S that would most probably provide a target knee angle of the leg LG as a function of time during one walking step.

The sensor providing the feedback signal FB may in addition to or as an alternative to the shown goniometer SN be a sensor placed in the product SP, such as the illustrated pressure sensors on FIGS. 2 and 3, or such sensors may be in the form of simple electric switches mounted between two layers of the product SP. One major purpose of the feedback FB to the control algorithm CA is to provide feedback FB regarding if the foot FT touches the ground or not, so as to provide valuable information to the control algorithm CA with respect to determining when a gait cycle is initiated. It is to be understood that several sensor types may be used to derive a feedback signal which represents a parameter describing the position and/or movement of the leg during the gait. This could be accelerometers, tiltsensors and gyroscopes that are used for estimating the position of the leg and thereby joint angles. E.g. a camera can be used to capture digital images of the leg during the patient walking. Via appropriate image analysis processing on the image, a suitable feedback can be derived, e.g. in the form of one or more of hip, knee, and ankle joint angles and foot distance to ground, or in the form of a more complex parametric description of the leg movement. However, in case only sensor(s) are mounted in the product SP, only one single electric interface is required to connect both stimulation electrodes as well as sensors providing the feedback FB to the processor unit PU.

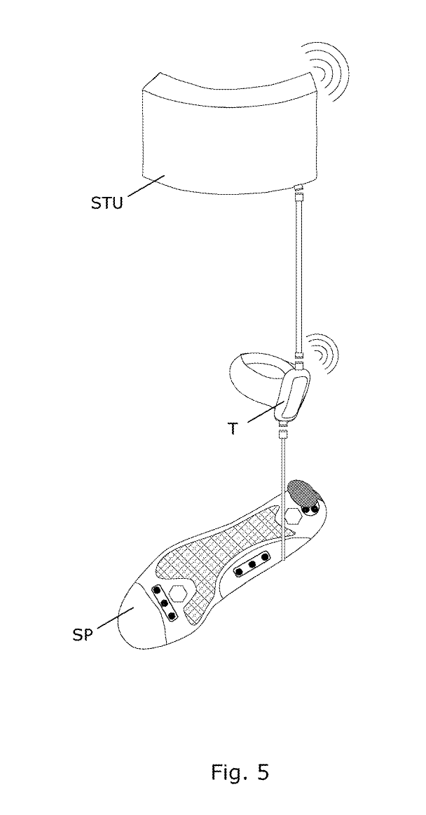

FIG. 5 shows a drawing of equipment for a FET system including an insole product SP, here illustrated as the one from FIGS. 3a, 3b. The insole SP is via its electric interface connected to a transmitter T which has a strap for fastening around the person's lower leg. The transmitter is connected to transmit a wireless RF signal indicative of the pressure sensed by the pressure sensors in the insole, (and potentially also from sensors mounted together with the lower leg transmitter e.g. accelometers, tiltsensors, and gyroscopes) and thus such signal can be used as input to a stimulation unit STU or a control unit, e.g. a laptop computer, in a reflex-based gait therapy system. A stimulation unit STU is also connected via a wire to the electric interface I of the insole SP, and this unit generates the electric stimulation signal to the appropriate one(s) of stimulation electrodes on the insole SP in response to an incoming wireless RF signal from a control system, e.g. a laptop computer. The stimulation unit STU may be fastened to the person's waist belt or it may be fastened to the person with a dedicated strap.

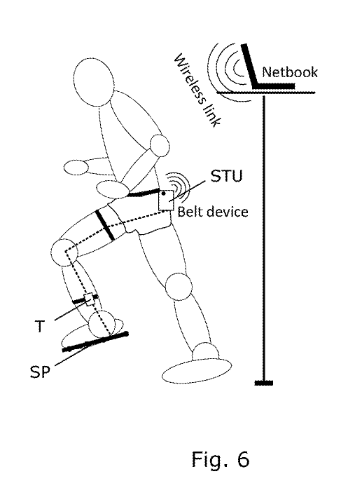

FIG. 6 illustrates the same equipment SP, T, STU as shown in FIG. 5, but here the equipment is sketched mounted on a person, and the wireless link to a netbook computer is also indicated. Thus, the control system of the FET system with feedback is implemented in software on the netbook computer, i.e. the control algorithm is run on the computer which wirelessly controls stimulation via the stimulation unit STU by transmitting a control signal which the unit STU translates into electric signal(s) which is applied to the appropriate one(s) of the stimulation electrodes.

It is to be understood that in all the illustrated embodiments, goniometers may be entirely replaced by sensor(s) in the form of accelerometer(s) and/or gyroscope(s). Especially, it may be preferred to build in such accelerometer(s) into a part of an insole.

To sum up, the invention provides a product, e.g. an insole for a shoe, fitting onto a person's foot sole based on a platform (SM) of a flexible and electrically isolating material. Multiple stimulation electrodes (S1, S2, S3) with adhesive stimulation areas of at least 1 cm² are spatially distributed on the platform (SM). A conducting and adhesive ground plane electrode (G) with at least an area of 10 times a stimulation electrode area is arranged for contacting the person's foot. An electric interface (I) comprising a plug or socket with externally accessible electrical connectors to allow external individual electrical access to the plurality of stimulation electrode areas (S1, S2, S3) and to the ground plane electrode (G). The electric interface is capable of handling at least a voltage of 200 V, preferably 400 V. Such product is suited for reflex-based gait therapy, where the person's withdrawal reflex is triggered by a painful stimulation of the person's foot sole. Many distributed stimulation electrodes allow individual fitting for optimal stimulation, and low cost sensors built into the product may be used to provide feedback regarding the gait phase, i.e. the product is a one-time use insole suited for easy and hygienic gait therapy in a clinic.

Although the present invention has been described in connection with preferred embodiments, it is not intended to be limited to the specific form set forth herein. Rather, the scope of the present invention is limited only by the accompanying claims.

In this section, certain specific details of the disclosed embodiments are set forth for purposes of explanation rather than limitation, so as to provide a clear and thorough understanding of the present invention. However, it should be understood readily by those skilled in this art, that the present invention may be practiced in other embodiments which do not conform exactly to the details set forth herein, without departing significantly from the spirit and scope of this disclosure. Further, in this context, and for the purposes of brevity and clarity, detailed descriptions of well-known device, circuits and methodology have been omitted so as to avoid unnecessary detail and possible confusion.

In the claims, the term “comprising” does not exclude the presence of other elements or steps. Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Reference signs are included in the claims however the inclusion of the reference signs is only for clarity reasons and should not be construed as limiting the scope of the claims.

1. A product shaped to fit onto a person's foot sole, the product comprising:
a platform of a flexible and electrically isolating material,
a plurality of separate stimulation electrodes each having an adhesive stimulation area of at least 1 cm², wherein the stimulation electrodes are spatially distributed on the platform so as to be able to stimulate different zones of the person's foot sole during normal use,
at least one electrically conducting and adhesive ground plane electrode for contacting the person's foot, wherein a total area of the ground plane electrode is at least 10 times an area of one stimulation electrode,
an electric interface comprising a plug or socket with externally accessible electrical connectors so as to allow external individual electrical access to the plurality of stimulation electrode areas and to the ground plane electrode, and
a set of electrical conductors for interconnecting the plurality of stimulation electrodes and the ground plane electrode with the electric connectors of the electric interface, wherein the set of electrical conductors are arranged to withstand a voltage of more than 200 V between the ground plane electrode and the plurality of stimulation electrodes.

2-27. (canceled)

28. The product according to claim 1, wherein the product is one of: a replaceable insole arranged to fit into a shoe, a slipper, or a shoe.

29. The product according to claim 1, wherein the product is a replaceable insole arranged to fit into a person's shoe, and wherein the platform comprises an upper layer and a lower layer with the set of electrical conductors arranged between the upper and lower layers, and wherein the stimulation electrodes extend through holes in the upper layer.

30. The product according to claim 29, wherein at least the upper layer is made of a reusable material.

31. The product according to claim 1, comprising a plurality of stimulation electrodes spatially distributed on the platform so as to enable stimulation of a plurality of different positions of each one of the person's foot sole areas: heel, fore foot, and mid foot.

32. The product according to claim 1, wherein the ground plane extends spatially so as to enable contact to a plurality of the person's foot regions: heel of foot sole, fore foot of foot sole, mid foot of foot sole, back of heel, or upper part of foot.

33. The product according to claim 1, comprising an additional platform element structurally linked to the platform, wherein the additional platform element comprises a stimulation electrode or a ground plane electrode in electric connection with said electric interface.

34. The product according to claim 33, wherein the additional platform element is connected to the platform so as to enable the stimulation electrode or the ground plane electrode to be in contact with an upper part of or a back of the heel of the person's foot when mounted for normal use.

35. The product according to claim 1, comprising a sensor arranged to provide a feedback indicative of a person's gait phase upon the person walking when wearing the product.

36. The product according to claim 35, wherein the sensor is electrically connected to one or more electrical connectors within said electric interface, and wherein at least one sensor is spatially arranged in relation to the platform so as to enable sensing of the person's heel touching the ground, when wearing the product.

37. The product according to claim 35, wherein the sensor comprises a plurality of spatially distributed sensor elements, in different regions of the platform so as to enable sensing of respective areas of the person's foot touching the ground, when wearing the product.

38. The product according to claim 1, wherein the platform forms part of a footwear.

39. A system arranged for gait training, the system comprising:
a product shaped to fit onto a person's foot sole, the product comprising
a platform of a flexible and electrically isolating material,
a plurality of separate stimulation electrodes each having an adhesive stimulation area of at least 1 cm², wherein the stimulation electrodes are spatially distributed on the platform so as to be able to stimulate different zones of the person's foot sole during normal use,
at least one electrically conducting and adhesive ground plane electrode for contacting the person's foot, wherein a total area of the ground plane electrode is at least 10 times an area of one stimulation electrode,
an electric interface comprising a plug or socket with externally accessible electrical connectors so as to allow external individual electrical access to the plurality of stimulation electrode areas and to the ground plane electrode, and
a set of electrical conductors for interconnecting the plurality of stimulation electrodes and the ground plane electrode with the electric connectors of the electric interface, wherein the set of electrical conductors are arranged to withstand a voltage of more than 200 V between the ground plane electrode and the plurality of stimulation electrodes, and
a stimulation unit arranged for connection to the electric interface of the product, wherein the stimulation unit is arranged to generate an electric stimulation voltage between the ground plane electrode and at least one of the stimulation electrodes, wherein the electric stimulation voltage is configured to induce a pain reaction on a person wearing the product.

40. The system according to claim 39, comprising:
a sensor arranged to sense a parameter representative of a movement of the person's leg and to generate a feedback signal accordingly, and
a processor unit operationally connected to the stimulation unit and to the sensor, the processor unit comprising a processor running a control algorithm so as to generate a control signal to the stimulation unit in response to the feedback signal.

41. A method of using the product set forth in claim 1 for functional electric stimulation of a foot of a person comprising:
providing the product of claim 1 to a foot of a person; and
providing electricity to the plurality of stimulation electrode areas and to the ground plane electrode of said product so as to provide functional electric stimulation of said foot of said person.

42. A method of using the product set forth in claim 1 to diagnose a central sensitization of a person in relation to chronic pain by assessing expanded reflex receptive fields via distributed electrical stimulation comprising:
providing the product of claim 1 to a foot of a person; and
providing electricity to the plurality of stimulation electrode areas and to the ground plane electrode of said product so as to provide functional electric stimulation of said foot of said person; and
diagnosing a central sensitization of said person to chronic pain by assessing expanded reflex receptive fields via distributed electrical stimulation.
